(19) United States
(12) Patent Application Publication
(10) Pub. No.: US 2019/0021598 A1
(43) Pub. Date: Jan. 24, 2019

(54) INTEGRATED CATHETER DEVICE FOR CARDIOVASCULAR DIAGNOSIS AND IMAGE PROCESSING SYSTEM

(71) Applicant: KOREA PHOTONICS TECHNOLOGY INSTITUTE, Gwangju (KR)

(72) Inventor: Joo Beom EOM, Gwangju (KR)

(21) Appl. No.: 16/127,755

(22) Filed: Sep. 11, 2018

Related U.S. Application Data
(63) Continuation of application No. PCT/KR2016/012681, filed on Nov. 4, 2016.

(30) Foreign Application Priority Data
Mar. 16, 2016 (KR) 10-2016-0031648

(51) Int. Cl. A61B 5/00; A61B 8/12; A61B 8/08; A61B 8/00

(52) U.S. Cl. CPC A61B 5/0035; A61B 5/0066; A61B 8/12; A61B 8/4416; A61B 5/0084; A61B 5/0095; A61B 5/6852; A61B 8/0891

(57) ABSTRACT

The present invention relates to an integrated catheter device for cardiovascular diagnosis and an image processing system using the same. According to an specific example of the invention, a cardiovascular image having a number of analytical aspects is obtained by acquiring an image in a cardiovascular region by one catheter using ultrasound, photoacoustic light, and near-infrared light, thereby enabling diagnosis of fibrosis of a blood vessel, atherosclerotic plaque, rupture prediction to be accurately performed.

Since the catheter device is provided with the DCF for guiding near-infrared light having a predetermined wavelength in a core portion and guiding photoacoustic light and ultrasonic wave used in a clad portion, it is possible to guide light having different wave-lengths to the sample through the same path simultaneously. Accordingly, it is advantageously possible to reduce the radius of the catheter device capable of acquiring the cardiovascular image having a number of analytical aspects, and to acquire ultrasound, acoustic wave and near-infrared light at the same time.

This application relates to International Application No. PCT/KR2016/012681, Filing Date of Nov. 4, 2016, which claims the benefit of Korean Application No. 10-2016-0031648, filed Mar. 16, 2016, each of which is incorporated by reference in their entirety.

### TECHNICAL FIELD

The present invention relates to an integrated catheter device for cardiovascular diagnosis and an image processing system using the same. More particularly, the present invention relates to for a technology for acquiring blood vessel images and a diameter of the blood vessel by measuring, acquiring a tomographic image of the inside of the blood vessel and an image showing contacts between a stent and a blood vessel, and acquiring blood concentration and lipid core area in the blood vessel, simultaneously by using one catheter.

### BACKGROUND ART

As the conventional art of a blood-vessel image catheter used for the diagnosis of cardiovascular diseases, intravascular ultrasound, intravascular near-infrared imaging, and intravascular optical coherence tomography have been commercialized and used in clinical practice.

An intravascular ultrasound technic is the art that a catheter-type device is inserted into blood vessels to acquire tomographic images of blood vessels. It is the most commonly utilized intravascular imaging technique in hospitals. Due to the use of ultrasonic technology, the resolution is as low as 100 μm, the contrast is low, and the image acquisition speed is as low as about 30 seconds.

In addition, intravascular near infrared (IR) imaging technology is a commercialized technology that uses near-infrared light to detect the presence of lipid in the inner wall of a blood vessel. Recently, it has been developed as a single catheter in combination with intravascular ultrasound.

Since this is the method using light, there is a problem that the signal sensitivity is not constant depending on the presence or absence of blood inside the blood vessel, the resolution is low, and the image acquisition speed is also slow because the image is acquired simultaneously with the intravascular ultrasound.

An intravascular optical coherence tomography technique used in an intravascular optical coherence tomography is the technology that a catheter-type device, in common with an intravascular ultrasound, transmits light to a blood vessel and analyzes the returned light to obtain a tomographic image of the blood vessel.

Since the speed of intravascular optical coherence tomography at the beginning was not as fast as the intravascular ultrasound, it has not been widely used. However, the recently developed second-generation intravascular optical coherence tomography technique has improved the speed by more than 10 times, thereby allowing an intravascular image to be photographed in a few seconds. In order to minimize the effect of blood because the light is used, images are obtained while flushing a solution of saline solution and a blood contrast medium.

With the improved resolution (˜10 μm) 10 times better than intravascular ultrasound, minute changes in the blood vessels can be observed. Recently, multifunctional imaging technology in combination with fluorescence imaging technology is being implemented at the laboratory level.

Accordingly, in order to acquire accurate images of fine cardiovascular blood vessels, it is necessary to insert/measure a catheter which is fine enough to be inserted in a blood vessel. This increases the risk of complications development, since the degree of agreement between acquired images in terms of a lesion is not accurate, cannot be used for emergency diseases such as AMI (acute myocardial infarction). Due to limitations of each imaging technique, it is difficult to determine a histological characteristic of a vulnerable atherosclerotic plaque under high risk. Therefore, early diagnosis/prevention of acute coronary artery disease is difficult.

In view of the aforementioned limitations, the applicant of the present invention has invented based on acquiring images in cardiovascular using ultrasound, photoacoustic light and near-infrared light by using one catheter.

### DISCLOSURE

### Technical Problem

Accordingly, the present invention has been made to resolve the above problems, and it is an object of the present invention to provide an integrated catheter device for cardiovascular diagnosis and an image processing system using the same, capable of displaying a high-resolution cardiovascular image with high efficiency by acquiring images inside of cardiovascular using one catheter.

### Technical Solution

In order to achieve the above object, an image processing system using a catheter device for cardiovascular diagnosis according to the present invention comprises an imaging device having an OCT image unit using near-infrared light for acquiring an image showing contacts between a stent and a blood vessel and an optical tomographic microstructure image in a blood vessel of which the thickness of the thin fibrous membrane can be measured, and an ultrasonic and photoacoustic image unit for acquiring an image of structure in a blood vessel, by using an ultrasonic wave, of which the inner diameter can be measured and for acquiring an image for measuring a liquid concentration and a lipid core area by using a photoacoustic light; a catheter device for transmitting near-infrared light and photoacoustic light and ultrasonic waves having a predetermined wavelengths of the imaging device to a sample; a photoprocessing device for concentrating the near-infrared light and photoacoustic light having a predetermined wavelengths generated by the catheter device, transmitting them to the catheter device, separating the near-infrared light, the acoustic wave and the ultrasonic wave having a predetermined wavelength returned back from the sample, and transmitting them to the imaging device.

Preferably, the OCT image unit may comprise a near-infrared light generator for generating light having a predetermined wavelengths; a first SMF (Single-mode fiber) coupler for guiding the light to a reference end through a first path; a double clad optical fiber coupler for receiving the light through a second path of the first SMF coupler, coupling and transmitting to the light processing device; and a second SMF coupler for coupling the light received at the double clad optical fiber coupler guiding the light reflected from the sample and the light reflected at the reference end, and transmitting the combined light to a detector, to obtain the OCT image.

Preferably, the ultrasound and photoacoustic image unit comprises a pulse laser generator for generating the photoacoustic light to be irradiated to a sample, that is transmitted to the catheter device through the double clad fiber coupler via the light processing device; a receiving end for receiving the acoustic waves by thermal energy generated by the irradiated light through the catheter device from the sample, and for delaying the photoacoustic light and then receiving the photoacoustic light as a reference signal; and a processing unit for processing the reference signal and the acoustic wave signal of the receiving end to obtain an IVPA image.

Preferably, the catheter device may further comprises DCF (Double Cladding Fiber) for guiding the light having a predetermined wavelengths provided from the light processing device to a sample; a lens for passing the light passed through the DCF; and a prism for refracting the light passed through the lens in order to irradiate it to a predetermined target position of the sample, and, wherein the DCF, the lens, and the prism are modularized as one to be provided into a single housing having a light source opening for scanning the light to the sample. And the housing may be provided with a quartz capillary tube for preventing the inflow of liquid.

Preferably, the catheter device may further comprise an ultrasonic transducer for receiving the acoustic wave provided from the sample with respect to the photoacoustic light generated in the ultrasonic wave and photoacoustic image unit and transmitting the received acoustic wave to the receiving end of the ultrasonic wave and the photoacoustic image unit.

In addition, the DCF may comprise a core portion for guiding near-infrared light of a predetermined wavelength of the OCT image unit to a sample and transmitting the light source reflected from the sample to the OCT image unit; and a clad portion for guiding the photoacoustic light of the pulse laser of the ultrasound and photoacoustic image unit to the sample.

Preferably, the ultrasound and photoacoustic image unit may further comprise an ultrasound generator for generating ultrasound having the predetermined frequency and transmitting the generated ultrasound to the sample through the ultrasound transducer in such a way the ultrasonic wave reflected from the sample is received by the receiving end through the ultrasound transducer of the catheter device and transmitted to the processing unit.

Another aspect of the present invention comprises DCF (Double Cladding Fiber) for guiding a light having a predetermined wavelengths generated in a imaging device to a sample; a lens for passing the light passed through the DCF; and a prism for refracting the light passed through the lens in order to irradiate it to a predetermined target position of the sample, and, wherein the DCF, the lens, and the prism are modularized as one to be provided into a single housing having a light source opening for scanning the light with the sample.

Preferably, the housing may be provided with a quartz capillary tube for preventing the inflow of liquid.

The DCF may include two channels comprising a core portion for guiding a near-infrared light having a predetermined wavelength to a sample and transmitting the light reflected from the sample to the imaging device, and a clad portion for guiding the photoacoustic light generated in the imaging device to the sample.

### Advantageous Effects

According to the present invention, a cardiovascular image having a number of analytical aspects is obtained by acquiring an image in a cardiovascular region by cooperating ultrasound, photoacoustic light, and near-infrared light using one catheter, thereby enabling diagnosis of fibrosis of a blood vessel, atherosclerotic plaque, rupture prediction to be accurately performed.

According to the present invention, since the catheter device is provided with the DCF for guiding near-infrared light having a predetermined wavelength of the imaging device to a sample in a core portion of the catheter device and guiding the photoacoustic light of the pulsed laser to a sample in a clad portion, it is possible to guide the lights having different wave-lengths to the sample through the same path simultaneously by using a single DCF. Accordingly, it is advantageously possible to reduce the radius of the catheter device capable of acquiring the cardiovascular image having a number of analytical aspects, and to acquire ultrasound, acoustic wave and near-infrared light at the same time.

### DESCRIPTION OF DRAWINGS

The accompanying drawings attached to this specification are only intended to illustrate preferred embodiments of the present invention to give a better understanding of the technical idea of the present invention with the description of the invention as in the following. Accordingly the illustrative drawings should not be construed as any limitation to the present invention.

FIG. 1 is a view illustrating a configuration of an image processing system using an integrated catheter for cardiovascular diagnosis according to an embodiment of the present invention.

FIG. 2 is a view illustrating a configuration of a catheter device of an image processing system using an integrated catheter device for cardiovascular diagnosis according to an embodiment of the present invention.

FIG. 3 illustrates a guiding state of light of a catheter device of an image processing system using an integrated catheter for cardiovascular diagnosis according to an embodiment of the present invention.

FIG. 4 is a cross-sectional view of a catheter device of an image processing system using an integrated catheter for cardiovascular diagnosis according to an embodiment of the present invention.

FIG. 5 is a view illustrating another example of a catheter device of an image processing system using an integrated catheter for cardiovascular diagnosis according to an embodiment of the present invention.

FIG. 6 is a diagram illustrating a detailed configuration of an image processing system using an integrated catheter for cardiovascular diagnosis according to an embodiment of the present invention.

FIGS. 7A to 7C are views illustrating an operation state of a catheter device of an image processing system using an integrated catheter for cardiovascular diagnosis according to an embodiment of the present invention.

FIGS. 8A to 8C are views illustrating images obtained in an image processing system using an integrated catheter for cardiovascular diagnosis according to an embodiment of the present invention.

### BEST MODE

Hereinafter, embodiments of the present invention will be described in detail with reference to the accompanying drawings so that those skilled in the art can easily operate the present invention. However, the present invention may be embodied in many different forms and should not be construed as limited to the embodiments set forth herein. In order to clearly illustrate the present invention, parts not being related to the description are omitted, and similar parts are denoted by same reference numerals throughout the specification.

Throughout the specification, when a part is referred to as being “connected” to another part, it includes not only “directly connected” but also “electrically connected” with another part in between. When a part is referred to as “including(comprising)”, it means that it can include(comprise) further parts as well, without excluding other parts unless specifically stated otherwise.

Hereinafter, a circuit service method according to an embodiment of the present invention will be described with reference to the accompanying drawings.

The image processing system S according to the embodiment of the present invention can provide a comprehensive intra-cardiovascular image by one catheter using ultrasound, photoacoustic light, and a near-infrared light. The comprehensive intra-cardiovascular image is visualized by processing an optical tomographic image obtained on the basis of ultrasound, acoustic wave, and near-infrared light reflected from a sample and various cardiovascular images at the same time.

Accordingly in the present invention, a cardiovascular image with high efficiency by one catheter having a double clad optical fiber coupler can be acquired and displayed. The ultrasonic wave, photoacoustic light, and near-infrared light are transmitted to a blood vessel by the catheter, and then optical tomographic image analysis, acoustic wave analysis, and ultrasonic wave analysis are processed based on the ultrasonic wave, acoustic wave, and near-infrared light returned from the blood vessel.

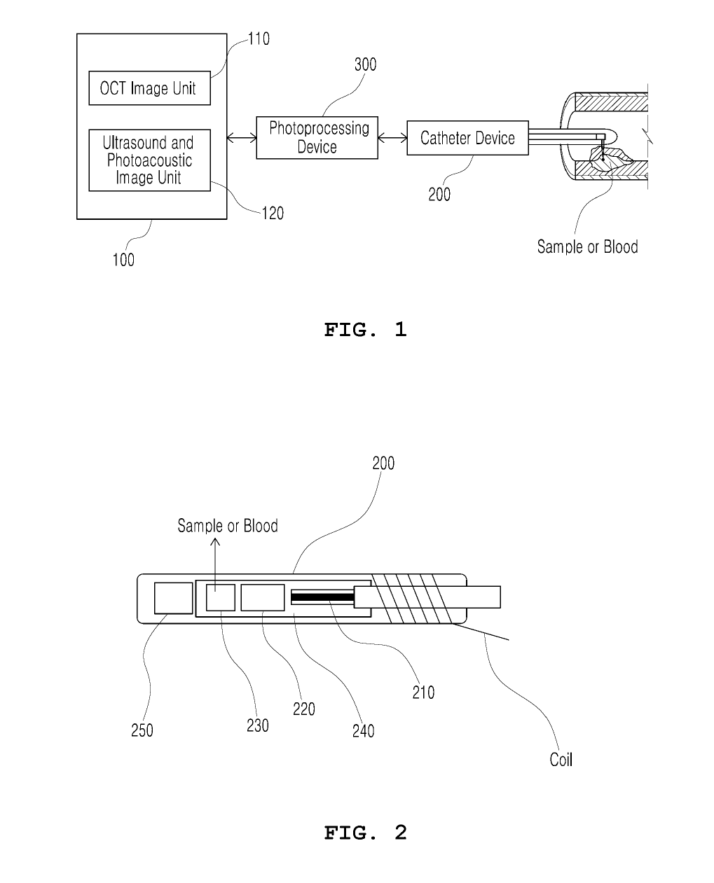

FIG. 1 is a view illustrating a configuration of an image processing system acquiring images of a multi-analyzing aspect from light collected by using an integrated catheter for cardiovascular diagnosis according to an embodiment of the present invention. With reference to FIG. 1, the following can be included: an imaging device 100 for generating and transmitting light and ultrasonic wave of a number of wavelengths, and visualizing a blood vessel based on the received light, ultrasonic signals and acoustic wave signals, a catheter device 200 for transmitting an ultrasonic wave and a light having a number of wavelengths to a blood vessel, and a light processing device 300 for combining or separating a ultrasonic wave and light having a number of wavelengths and transmitting them to the catheter device 200 and the imaging device 100. Here, the light having predetermined wavelengths may include photoacoustic light of the pulse laser and wavelength tunable near-infrared light. And the photoprocessing device 300 may include a function of transmitting near-infrared light and photoacoustic light and of connecting mechanically a fixed imaging device 100 with a rotating catheter device 200. Accordingly, the image processing function is not degraded even when the catheter device 200 is rotated.

The imaging device 100 includes an OCT image unit 110 for acquiring an IVOCT (intravascular optical coherence tomography) image, and may include an ultrasound and photoacoustic image unit 120 for acquiring an IVPA (Intra Vascular Photo Acoustic) image to measure a liquid concentration and a lipid core area in a blood vessel, an IVUS (Intravascular Ultra Sound) to measure the image of the entire blood vessel and the diameter of the inside of the blood vessel.

In the embodiment of the present invention, as the light, acoustic wave signal and ultrasonic signal provided from the single catheter device 200 are processed in the OCT image unit 110 and the ultrasound and photoacoustic image unit 120, the image of a blood vessel is acquired simultaneously with integrated processing at the same position.

The catheter device 200 is a tubular device for insertion into an organ having a body cavity or luminal cavity. The catheter device 200 constituting the image processing system S refers to a device for imaging the inside of a blood vessel by using a Double-Clad Fiber (hereinafter, DCF).

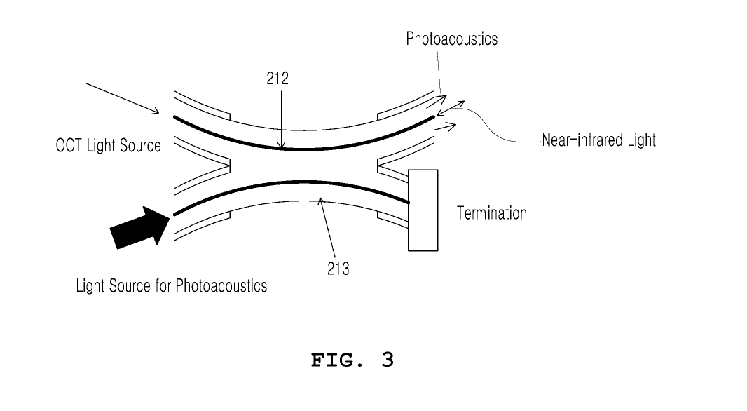

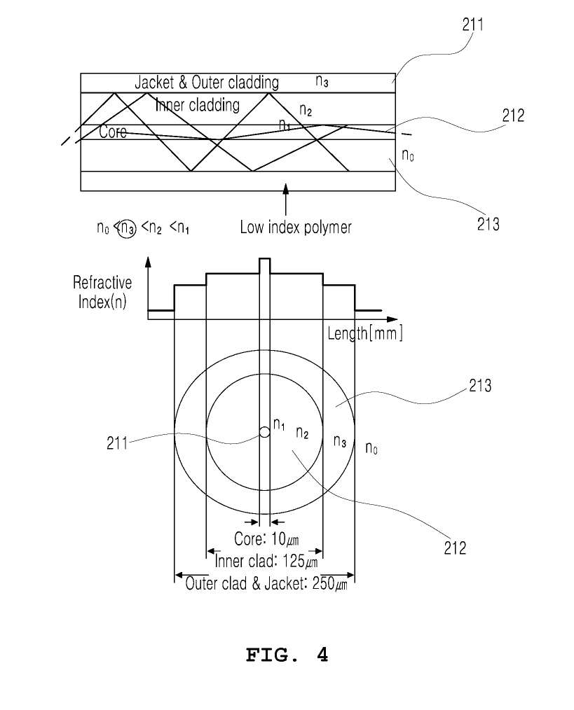

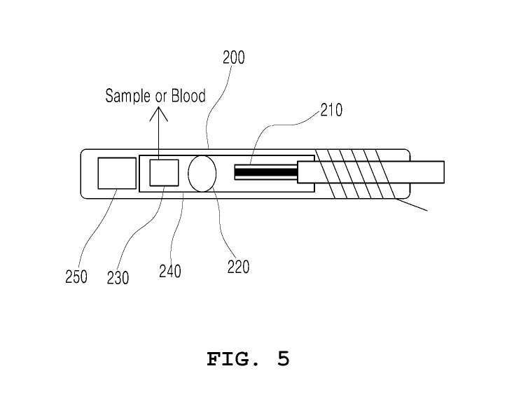

FIG. 2 is a view illustrating a configuration of the catheter device 200 illustrated in FIG. 1, FIG. 3 is a view illustrating a specific configuration of a DCF (Double Claddering Fiber) 210 illustrated in FIG. 2, FIG. 4 is a cross-sectional view of the DCF 210 illustrated in FIG. 2, and by referring to FIGS. 2 to 4, the catheter device can be equipped with the DCF 210 which transmits near-infrared light and photoacoustic light of the pulse laser to an inner blood vessel.

The DCF 210 is a path for moving the photoacoustic light and near-infrared light provided from the imaging device 100, and comprises a coating tube 211 surrounding the optical fiber and two channels, that is, a core portion 212 and a clad portion 213 as a path through which the light travels.

Accordingly, the core portion 212 can acquire an OCT image by using the OCT image unit 110 and the clad portion 213 can acquire an IVPA image by using the light source for photoacoustics of the ultrasound and photoacoustic image unit 120.

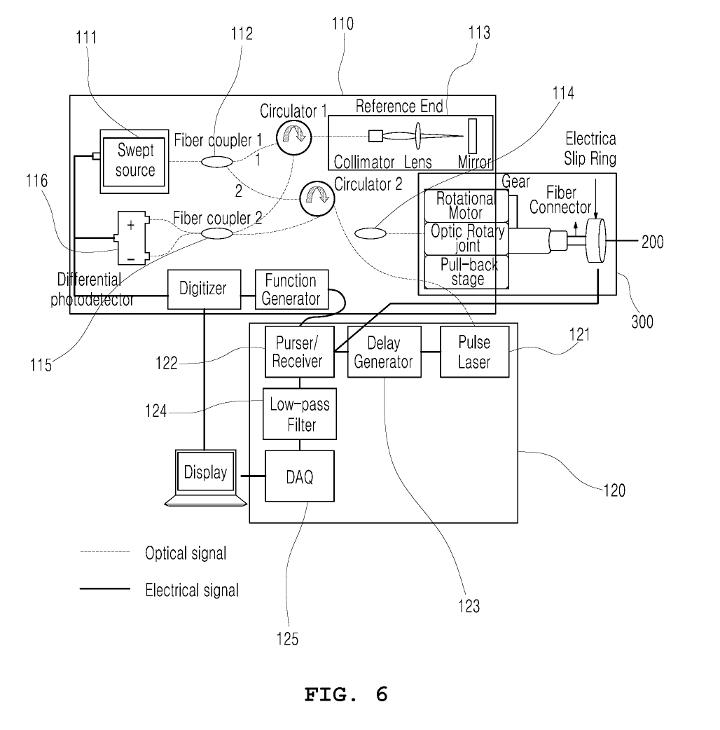

In addition, the catheter device 200 may further comprise a GRIN lens 220 based on the optical fiber of the DCF 210 to produce a very small catheter. Here, the GRIN lens 220 as a lens combining and separating light can obtain a cardiovascular image by transmitting the light source of the imaging device 100 to the DCF 210 and rotating together with the DCF 210. Although the GRIN lens is described as an example for convenience of explanation in the embodiment of the present invention, it may be replaced with a lens such as a single lens, a ball lens, a C-lens, a doublet lens, and a lens array, etc. At this time, the catheter device 200 using a ball lens instead of the GRIN lens is as shown in FIG. 5.

In addition, the catheter device 200 may further comprise a prism 230 that refracts the light source having passed through the GRIN lens 220 to a target position of a sample and transmits the light source to a target position.

In addition, the catheter device 200 is provided to package the DCF 210, the GRIN lens 220 and the prism 230 in one housing 240, wherein the housing 240 is a quartz capillary, that is, a glass tube, which can prevent the inflow of the external blood or liquid, and pass the light emitted to sample and the light reflected from the sample.

In addition, the catheter device 200 may further comprise an ultrasonic transducer 250 for emitting ultrasonic waves to the sample to acquire ultrasound images and transmitting the ultrasonic waves received from the sample to the ultrasound and photoacoustic image unit 120 of the imaging device 100. In addition, the ultrasonic transducer 250 may receive ultrasonic waves reflected from the sample and transmit the ultrasonic wave to the ultrasound and photoacoustic image unit 120.

In the exemplary embodiment of the present invention, a series of processes of acquiring an OCT image, an IVPA image, and an IVUS image by analyzing the received light, the pulse laser, and the ultrasonic wave using one catheter device 200 will be described in detail with reference to the drawings, wherein light, laser, and signal can be intermixed.

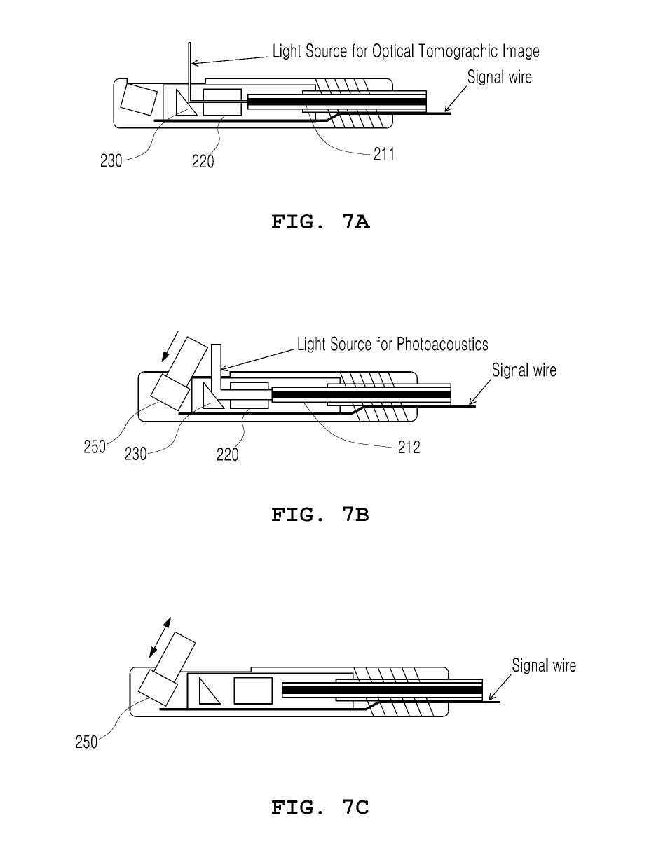

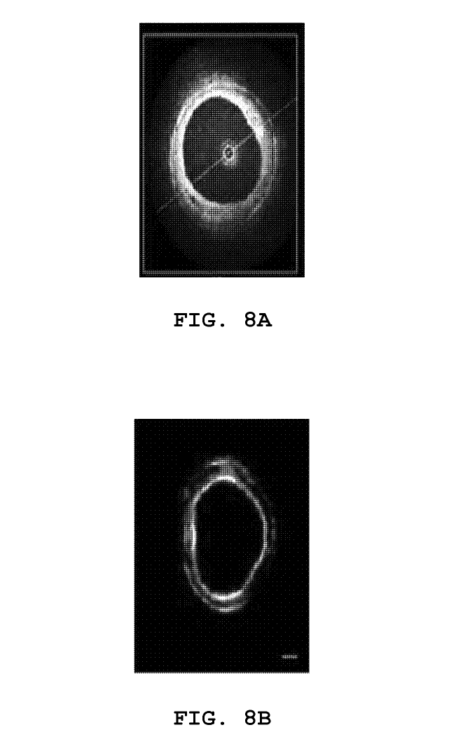

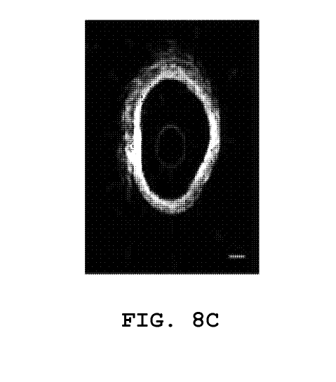

FIG. 6 is a view illustrating a detailed configuration of the imaging device 100 shown in FIG. 1, and FIGS. 7A to 7C are views illustrating a method of irradiating the ultrasonic wave, photoacoustic light, and near-infrared light received in the catheter device 200 shown in FIG. 1 to a sample or a blood vessel. FIG. 8A to 8C are views showing images acquired in the imaging device 100. Referring to FIGS. 6 to 8C, the imaging device 100 can be provided to irradiate an ultrasonic wave, a photoacoustic light, and a near-infrared light to a sample or a blood vessel through a single catheter. The irradiated ultrasonic wave, photoacoustic light, and near-infrared light can be guided through the catheter device 200 to detect an IVUS image, an IVPA image and an OCT image respectively.

That is, in the OCT image unit 110 of the imaging device 100, the light of the near-infrared light generator 111 for the tomographic microstructure image is guided to a first path of the first SMF coupler (Single mode fiber coupler) 112 and is provided to a reference end 113.

On the other hand, the light guided to a second path of the first SMF coupler 112 is condensed by the light processing device 300 and coupled to the core portion of the double clad optical fiber coupler 114 and guided to the catheter device 200. In this case, the near-infrared light generator 111 may be used as a device for generating a wavelength-tunable laser such as 1310 nm, 1060 nm, or 1550 nm.

That is, the light source of the near-infrared light generator 111 may be provided to the first SMF coupler 112 having two ports, and the light having passed through one port of the first SMF coupler 112 is transmitted to the reference end 113 through Circulator 1 and the light passing through the other port is transmitted to the double clad optical fiber coupler 114 via Circulator 2 and is transmitted to the sample by passing through the catheter device 200 via the core of the double clad optical fiber coupler 114.

And the light provided to the catheter device 200 sequentially passes through the core portion 211 of the DCF 210 of the catheter device 200, the GRIN lens 220, and the prism 230, as shown in FIG. 7A and is scanned to a sample or a blood vessel.

And the scanned light is emitted back through a path sequentially passing through the core portion 211 of the DCF 210, the GRIN lens 220, and the prism 230, and separated by the light processing device 300. And then it is transmitted to the detector 116 through the second SMF coupler 115 of the OCT image unit 110 after through Circulator 2 via the core of the double clad optical fiber coupler 114 of the OCT image unit 130. At this time, the light reflected by the reference end 113 is passed through Circulator 1 and transmitted to the detector 116 via the second SMF coupler 115 so that a signal can be detected.

That is, the second SMF coupler 115 generates an interference signal by combining the reference signal of the reference end 113 and the signal from the sample, the generated interference signal is converted into an electrical signal at the detector 116, and an optical tomographic micro-structure (OCT) image shown in FIG. 8A can be acquired through digital conversion and Fourier transformation thereafter.

Accordingly, tomographic imaging of the interior of a blood vessel can be acquired to determine whether a stent of the treatment current of the BVS (Bioresorbable Vascular scaffold stent) is closely attached to a blood vessel, whether a stent thrombosis has occurred, whether a soft tissue of a wall has been flaked, as well as the thickness of the thin fibrous membrane of the blood vessel, etc.

On the other hand, the photoacoustic light generated by the pulse laser generator 121 of the ultrasound and photoacoustic image unit 120 is coupled to the clad portion of the double clad optical fiber coupler 114 of the OCT image unit 110, and guided to the catheter device 200. And the pulse laser for photoacoustics simply functions to transmit a light source, thereby allowing transmission in multimode. That is, the core of the double clad optical fiber coupler 114 operates in single mode while the clad operates in a multimode.

The photoacoustic light guided to the catheter device 200 sequentially passes through the clad portion 212 of the DCF 210, the GRIN lens 220, and the prism 230, to scan a sample or a blood vessel as shown in FIG. 7B.

At this time, the light incident from the sample generates thermal energy, a acoustic wave is generated from the generated thermal energy, and the generated acoustic wave is transmitted through the ultrasonic transducer 250 of the catheter device 200 and detected as a signal at the receiving end 122 of the ultrasonic wave and photoacoustic image part 120. At this time, the photoacoustic light generated by the pulse laser generator 121 is provided to the receiving end 122 via the delaying unit 123, and the reference signal of the delaying unit 123 and the acoustic signal of the ultrasonic wave provided from the ultrasonic transducer 250 are filtered via the low pass filter 124 and the IVPA image shown in FIG. 8B can be acquired for measuring thickness of a lipid core and blood components in DAQ (Data Acquisition) 125.

On the other hand, as illustrated in FIG. 7C, an ultrasonic wave of the ultrasonic wave generator of the ultrasound and photoacoustic image unit 120 is irradiated to the sample or the blood vessel via the ultrasonic transducer 250 of the catheter device 200, the ultrasonic wave reflected from the sample or the blood vessel is received at the receiving end 122 of the ultrasound and photoacoustic image unit 120, the received ultrasonic wave is filtered through the low pass filter 124, and the IVUS image shown in FIG. 8C for measuring cardiovascular internal structure and diameter and the like can be acquired at the DAQ (Data Acquisition).

Description of the invention described above will be appreciated that is for illustrative purposes. One of ordinary skill in the art without changing the technical spirit or essential features of the present invention easily deformed is possible in other specific forms will be. Thus the embodiments described above are only to be understood as illustrative and non-restrictive in every respect. For example, the components that are described in one-piece can be performed with the embodiment may be distributed, combined also it has been described as distributed components, While the present invention has been described in connection with a preferred embodiment, it is to be understood by a person of ordinary skill in the pertinent art that the invention is not limited to the disclosed embodiments, but can be easily modified to other specific form without changing the technical spirit or essential characteristics of the present invention. Accordingly, the embodiments described above are only to be understood as illustrative and non-restrictive in every aspect. For example, the components that are described in one-piece can be performed in such a way that each component is dispersed, and the dispersed components can be performed as in a combined group.

The scope of the invention is to be defined by the accompanying claims rather than the above description and all such modifications as derived from the meaning and range and equivalents of the claims should be included in the scope of the invention.

### INDUSTRIAL APPLICABILITY

By one catheter using ultrasound, photoacoustic light and near-infrared light, an image in a cardiovascular region can be obtained. The obtained cardiovascular image having a number of analytical aspects is utilized for the diagnosis of fibrosis of a blood vessel, atherosclerotic plaque, and rupture prediction. Since the catheter device is provided with the DCF for guiding near-infrared light having a predetermined wavelength of the imaging device to a sample in a core portion of the catheter device and guiding the photoacoustic light of the pulsed laser to a sample in a clad portion, it is possible to guide simultaneously the lights having different wave-lengths to the sample through the same path by using a single DCF. Accordingly, it is advantageously possible to reduce the radius of the catheter device capable of acquiring the cardiovascular image having a number of analytical aspects. Accordingly, it is possible to make a great progress with respect to an integrated catheter device for cardiovascular diagnosis and an image processing system using the same, in terms of the accuracy of operation, reliability of the system, and performance efficiency. Furthermore the sales possibility of the medical equipment is increased, and it is practically possible to carry out clearly. Thus, the present invention can be regarded to have industrial applicability.

1. An image processing system using an integrated catheter for cardiovascular diagnosis comprising:
an imaging device having an OCT image unit using near-infrared light for acquiring an image showing contacts between a stent and a blood vessel and an optical tomographic microstructure image in a blood vessel of which the thickness of the thin fibrous membrane can be measured, and an ultrasonic and photoacoustic image unit for acquiring an image of structure in a blood vessel, by using an ultrasonic wave, of which the inner diameter can be measured and for acquiring an image for measuring a liquid concentration and a lipid core area by using a photoacoustic light;
a catheter device for transmitting the near-infrared light and photoacoustic light and ultrasonic waves having a predetermined wavelengths of the imaging device to a sample; and
a photoprocessing device for concentrating the near-infrared light and photoacoustic light having a predetermined wavelengths generated by the catheter device, transmitting them to the catheter device, separating the near-infrared light, the acoustic wave and the ultrasonic wave having a predetermined wavelength returned back from the sample, and transmitting them to the imaging device.

2. The image processing system according to claim 1, wherein the OCT image unit comprises:
a near-infrared light generator for generating light having a predetermined wavelengths;
a first SMF (Single-mode fiber) coupler for guiding the light to a reference end through a first path;
a double clad optical fiber coupler for receiving the light through a second path of the first SMF coupler, coupling and transmitting to the light processing device; and
a second SMF coupler for coupling the light received at the double clad optical fiber coupler guiding the light reflected from the sample and the light reflected at the reference end, and transmitting the combined light to a detector, to obtain the OCT image.

3. The image processing system according to claim 2, wherein the ultrasound and photoacoustic image unit comprises:
a pulse laser generator for generating the photoacoustic light to be irradiated to a sample, that is transmitted to the catheter device through the double clad fiber coupler via the light processing device;
a receiving end for receiving the acoustic waves by thermal energy generated by the irradiated light through the catheter device from the sample, and for delaying the photoacoustic light and then receiving the photoacoustic light as a reference signal; and
a processing unit for processing the reference signal and the acoustic wave signal of the receiving end to obtain an IVPA image.

4. The image processing system according to claim 3, wherein the ultrasound and photoacoustic image unit further comprises an ultrasound generator for generating ultrasound having the predetermined frequency so as to transmit to the sample, and the ultrasonic wave reflected from the sample is received at the receiving end and then transmitted to the processing unit.

5. The image processing system according to claim 4, wherein the catheter device comprises:
DCF (Double Cladding Fiber) for guiding the light having a predetermined wavelengths provided from the light processing device to a sample;
a lens for passing the light passed through the DCF; and
a prism for refracting the light passed through the lens in order to irradiate it to a predetermined target position of the sample,
and, wherein the DCF, the lens, and the prism are modularized as one to be provided into a single housing having a light source opening for scanning the light to the sample.

6. The image processing system according to claim 5, wherein the housing is provided with a quartz capillary tube for preventing the inflow of liquid.

7. The image processing system according to claim 6, wherein the catheter device further comprises an ultrasonic transducer for receiving the acoustic wave reflected from the sample with respect to the light having a predetermined wavelength and transmitting to the receiving end of the ultrasonic wave and the photoacoustic image unit.

8. The image processing system according to claim 5, wherein the DCF comprises two channels having:
a core portion for guiding a light having a predetermined wavelength of the OCT image unit to the sample and transmitting the light reflected from the sample to the OCT image unit;
a clad portion for guiding a photoacoustic light of the ultrasound and photoacoustic image unit to the sample.

9. An integrated catheter device for cardiovascular diagnosis comprising:
DCF (Double Cladding Fiber) for guiding a light having a predetermined wavelengths generated in a imaging device to a sample;
a lens for passing the light passed through the DCF; and
a prism for refracting the light passed through the lens in order to irradiate it to a predetermined target position of the sample,
and, wherein the DCF, the lens, and the prism are modularized as one to be provided into a single housing having a light source opening for scanning the light with the sample.

10. The integrated catheter device according to claim 9, wherein the housing is provided with a quartz capillary tube for preventing the inflow of liquid.

11. The integrated catheter device according to claim 10, wherein the catheter device further comprises an ultrasonic transducer for an ultrasonic wave provided from the imaging device to the sample and transmitting the ultrasonic wave of the sample to the ultrasound and photoacoustic image unit.

12. The integrated catheter device according to claim 11, wherein the ultrasonic transducer is provided to receive an acoustic wave reflected from the sample with respect to the photoacoustic light of the imaging device, and transmit it to the imaging device.

13. The integrated catheter device according to claim 9, wherein the DCF comprises two channels having:
a core portion for guiding a near-infrared light having a predetermined wavelength generated in the imaging device to the sample and transmitting the near-infrared light reflected from the sample to the imaging device; and
a clad portion for guiding the photoacoustic light generated in the imaging device to the sample.
